(19) United States
(12) Patent Application Publication
(10) Pub. No.: US 2008/0208022 A1
(43) Pub. Date: Aug. 28, 2008

(54) Laser Optical Feedback Tomography Sensor and Method

(75) Inventors: Sascha Kruger, Hamburg (DE); Tim Nielsen, Hamburg (DE)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONICS, N.V., EINDHOVEN (NL)

(21) Appl. No.: 11/916,545

(22) PCT Filed: Jun. 1, 2006
(86) PCT No.: PCT/IB2006/051759

§ 371 Date: Dec. 5, 2007

(30) Foreign Application Priority Data
Jun. 7, 2005 (EP) 05104936.9

(51) Int. Cl. A61B 5/1459

(52) U.S. Cl. 600/342

(57) ABSTRACT

The invention relates to a modified Laser Optical Feedback Tomography sensor (10) which comprises an evaluator (16) for the determination of an object velocity (vz) relative to the sensor (10). The primary optical frequency (fₒ) of light emitted by a laser (11) is shifted by a first frequency shift F in a frequency shifter (13) and focused into an investigation region (3). A moving object (2) in said region produces an additional Doppler frequency shift ΔF in the light sent back from the investigation region (3) which is re-injected into the laser (11). Resulting intensity oscillations of the laser (11), which critically depend on the shifted frequency of the re-injected light, are detected by a detector (15). Finally, the evaluator (16) coupled to the detector (15) determines from the observed oscillations the Doppler frequency shift ΔF and therefrom the moving velocity (V_{z}) of the object (2).

The invention relates to a modified Laser Optical Feedback Tomography sensor, an interventional instrument provided with such a sensor, a method for the determination of the relative velocity between an object and an instrument, and a scanning mechanism for selectively directing a radiation beam from an interventional instrument into the surrounding medium.

It is known in medical diagnostics to measure blood flow velocity based on a Doppler shift in reflected ultrasound waves. Such measurements are however affected by artifacts arising near the ultrasound transducer and by scattering from metal components, for example from a stent implanted into the vessel system for the treatment of a stenosis. In order to improve the accuracy of ultrasound measurements, the DE 38 39 649 A1 proposes to generate gas bubbles in the blood stream, which is however a rather complicated procedure.

Based on this situation it was an object of the present invention to provide means for a reliable determination of the relative velocity between an object and an instrument that are particularly suited for an application in medical interventions.

This object is achieved by a sensor according to claim 1, by an interventional instrument according to claim 5, by a method according to claim 6, and by a scanning mechanism according to claim 11. Preferred embodiments are disclosed in the dependent claims.

According to its first aspect, the invention relates to a sensor for the determination of the relative velocity of a moving object, more particularly to a Laser Optical Feedback Tomography (LOFT) sensor. The sensor comprises the following components:

A laser source for emitting a radiation beam at a primary optical frequency.

A frequency shifter for shifting the primary optical frequency f₀ of the radiation beam by a first frequency shift F,

Optics for irradiating an investigation region of a medium to be studied with radiation of the shifted frequency (f₀+F) and for re-injecting into the laser light sent back from the investigation region.

A detector for detecting the disturbance brought to the laser emission by the re-injected light.

An evaluator coupled to the detector and adapted to estimate the relative velocity of moving objects in the investigation region based on the detected disturbances and the first frequency shift F.

The technology of Laser Optical Feedback Tomography or LOFT is known from literature (cf. U.S. Pat. No. 6,476,916 B1; E. Lacot, R. Day, F. Stoeckel: “Laser Optical Feedback Tomography”, Opt. Lett., 24(11), June 1999, pages 744-746; E. Lacot, R. Day, F. Stoeckel: “Coherent Laser Detection By Frequency-Shifted Optical Feedback”, Phys. Rev. A, 64, 043815; these documents are incorporated into the present application by reference). LOFT is based upon the effect of ultra-short laser cavities (≈1 mm) to serve as temporal and spatial filter in frequency modulated re-injection mode. The laser serves as light source and coherent detector in one. A signal-amplification of up to 6 orders of magnitude has been reported. Strongly scattered photons are vetoed, because they do not match the coherence criterion. Only photons scattered from inside the laser focus are re-injected into the laser by mode matching. This technique is capable of imaging in highly scattering media (turbid media) with high spatial resolution.

Based on the aforementioned LOFT technology, the present invention provides an enhanced sensor that allows the determination of the velocity of an object relative to the sensor or at least the determination of one component of said velocity in a predetermined direction. According to the invention, the known LOFT sensor is extended by an evaluator which is capable of inferring the relative velocity of a moving object based on (i) the disturbances in the laser emissions detected by the detector and caused by the re-injected light, and (ii) the first frequency shift F that is introduced by the frequency shifter in the original laser light.

Regarding the standard LOFT components of the sensor described in this application, all modifications known from literature can be realized, too. This comprises for example the possibility to scan the frequency of the radiation emitted by the laser; to use a photodetector followed by a synchronous detection system in the detector; to excite electro-optical or acousto-optical effects in the frequency shifter; to arrange several sensors in an array and the like.

The evaluator of the sensor is preferably adapted to estimate the relative velocity of a moving object based on a second frequency shift that is caused by said object in the light sent back from the investigation region. Said second frequency shift is typically caused by the Doppler effect. Therefore, a known relation exists between the speed of light c in the respective medium, the frequency (f₀+F) of said light, the velocity component v_{z} of the object in the direction of the light propagation, and the resulting Doppler frequency shift ΔF. As the Doppler frequency shift ΔF in the light sent back from the investigation region can be inferred from the disturbances introduced in the laser by said light and as c, f₀, and F are known, the Doppler relation allows the estimation of the relative object velocity v_{z}.

According to a preferred embodiment, the frequency shifter is adapted to selectively generate different first frequency shifts F. Thus different set points of F can be established according to the range and/or direction of expected object velocities. Moreover, the frequency shifter may be adapted to scan (continuously or stepwise) a range of different first shifting frequencies F and thus a range of relative object velocities.

According to another embodiment of the invention, the optics of the sensor is adapted to move the investigation region through the medium to be studied, wherein the investigation region is determined by the focus volume of the light emitted from the sensor into said medium. By moving the investigation region through the medium, it is possible to scan one-, two- or even three-dimensional sub-regions of the medium.

The invention further relates to a minimal invasive interventional instrument, in particular to a catheter or an endoscope, wherein said instrument is provided with a modified LOFT sensor of the kind described above. The sensor of this instrument can be used like the known LOFT sensor, allowing to look ahead into turbid media like blood or tissue. In addition, the sensor can be used to determine the relative velocity between the instrument and an object in the medium surrounding the instrument (or its sensitive tip). Thus it may for example be possible to measure blood flow velocity with the instrument or to determine the moving velocity of the instrument relative to an organ in order to assist navigation of the instrument in a body volume.

The invention further relates to a method for the determination of the velocity of an object relative to an instrument, said method comprising the following steps:

Providing the instrument, for example a catheter or an endoscope, with a LOFT sensor comprising a laser, particularly a modified LOFT sensor of the kind described above.

Irradiating the object with radiation that is shifted from the primary optical frequency f₀ of the laser by a first frequency shift F,

Re-injecting into the laser light sent back from the investigation region.

Detecting the disturbance brought to the laser emission by the re-injected light.

Estimating the relative velocity of the object in the investigation region based on the detected disturbances and the first frequency shift F.

The method comprises in general form the steps that can be executed with a sensor of the kind described above. Therefore, reference is made to the preceding description for more information on the details, advantages and improvements of that method.

According to a preferred embodiment of the method, the relative velocity of the object is determined based on a second (Doppler) frequency shift caused by said object in the light sent back from the investigation region.

Moreover, the first frequency shift F introduced by the frequency shifter differs by a certain frequency gap from a relaxation frequency Fᵣₑₗₐₓ of the laser. Though the standard LOFT case of a frequency gap of width zero (i.e. F=Fᵣₑₗₐₓ) shall be comprised by the present invention, said gap is typically different from zero (i.e. F≠Fᵣₑₗₐₓ). Possible choices of F will be explained in more detail in connection with further embodiments of the invention. In an optional variant of the aforementioned method, the first frequency shift F is preferably chosen such that expected second (Doppler) frequency shifts caused by moving objects to be monitored are smaller than a frequency gap between the chosen first frequency shift F and the relaxation frequency Fᵣₑₗₐₓ of the laser. With other words, the Doppler shifted frequency of the light coming back from the investigation region is always between the frequency of the light sent into the investigation region and a resonance frequency that would yield a maximal gain when re-injected into the laser. The disturbances introduced into the laser emission by the re-injected light will therefore depend uniquely on the Doppler frequency shift, thus allowing to determine said Doppler shift uniquely.

In an optional embodiment of the method, the first frequency shift F that is introduced into the original laser light is scanned through a predetermined range. This allows the scanning of object velocities to be measured.

According to a preferred application of the method, the instrument is navigated relative to the object, for example by continuous measurements (and integrations) of the relative velocity between object and instrument. The instrument may particularly be a catheter or an endoscope, and the object the vessel system or an organ (e.g. the heart) of a patient. The navigation may be assisted by a static or dynamic roadmap of the object. An advantage of this approach is that common movements of instrument and object (e.g. due to heart beat or patient movements) are automatically compensated.

The invention further relates to a scanning mechanism for selectively directing a radiation beam from an interventional instrument (e.g. a catheter or an endoscope) into the surrounding medium (e.g. blood or tissue). Said scanning mechanism may particularly be applied in the optics of a LOFT sensor of the kind described above. The scanning mechanism comprises a remotely movable mirroring element (for example a simple planar mirror) that is arranged at the light outlet of the instrument.

Preferably the mirroring element can be shifted along the propagation axis of the light incident on said mirroring element, and/or it can be rotated about the aforementioned axis, and/or it can be rotated about an axis that is perpendicular to the aforementioned axis of incident light. If all these possibilities of movement are realized, a radiation beam can be directed or focused to any point in a certain region ahead of the light outlet.

The scanning mechanism may be constructed in various different ways. Preferably, the mirroring element is mounted between two carriers that can be axially shifted relative to each other. Due to its contact to both carriers, the mirroring element will then be tilted during such a relative axial shifting movement.

According to a further development of the aforementioned embodiment, the carriers can be commonly (simultaneously) rotated about their body axis thus forcing the mirroring element to rotate with them.

The carriers may optionally be constituted by two concentric tubes which are preferably embedded in a third outer tube. Such a design is particularly suited for catheter applications. The innermost tube (first carrier) preferably has a window through which the mirroring element can contact the radially next tube of the arrangement (i.e. the second carrier).

These and other aspects of the invention will be apparent from and elucidated with reference to the embodiment(s) described hereinafter.

In the following the invention is described by way of example with the help of the accompanying drawings in which:

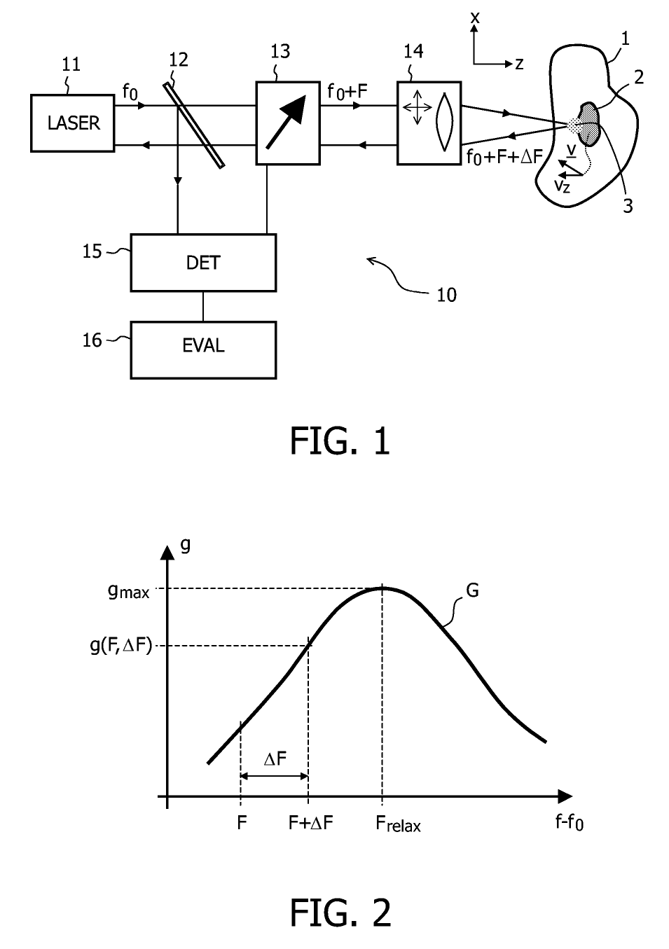

FIG. 1 shows a principal sketch of a modified LOFT sensor according to the present invention;

FIG. 2 illustrates the relation between the gain of a LOFT sensor and the frequency of re-injected light;

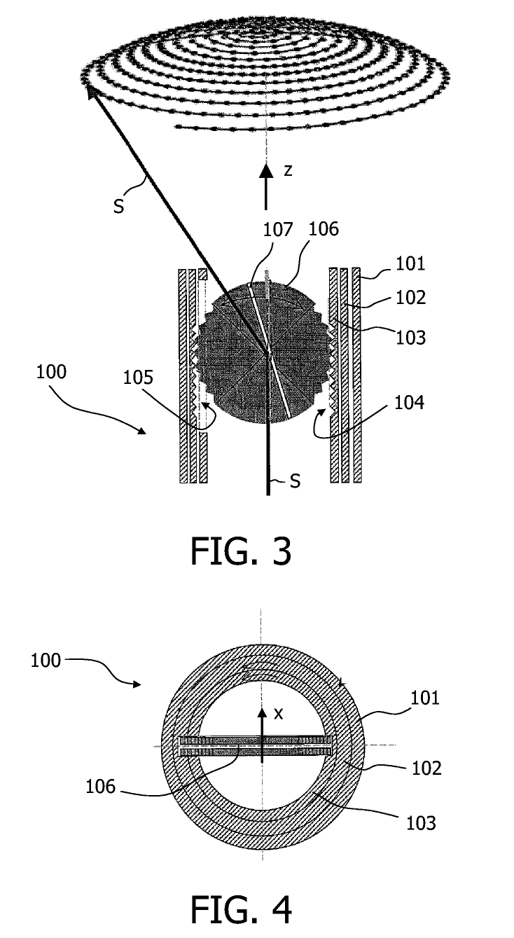

FIG. 3 shows a section through a scanning mechanism according to the present invention;

FIG. 4 shows a view in z-direction onto the mechanism of FIG. 3.

In FIG. 1, the principal construction of a LOFT sensor 10, denominated here as modified LOFT sensor 10, and its application to the measurement of the moving velocity v of an object 2 (e.g. a blood cell) in a medium 1 (e.g. blood) is shown. Many components of the LOFT sensor 10 correspond to those of usual LOFT sensors. For a detailed description of these components, reference is therefore made to the respective literature (e.g. U.S. Pat. No. 6,476,916 B1).

The LOFT sensor 10 comprises laser 11, for instance a class B laser, emitting a laser beam at a primary frequency f₀. Said laser beam partially passes a semi-reflective mirror 12 and enters a frequency shifter 13. In the frequency shifter 13, the frequency f₀ of the radiation beam is altered by a first frequency shift F to the value (f₀+F). The frequency shifter 13 may for example exploit electro-optical effects. Preferably, the first frequency shift F differs from a relaxation frequency Fᵣₑₗₐₓ of the laser (11) by a predetermined frequency gap.

The radiation beam of shifted frequency (f₀+F) next enters the optics 14 which comprises lenses and the like which focus the radiation into an investigation region 3 inside the medium 1 to be studied. As shown in the Figure, said investigation region 3 may particularly contain the surface of the object 2 moving with velocity v inside the medium.

At least a part of the incident light is reflected by the object 2 towards the optics 14. Due to the movement of the object 2 with the velocity component v_{z} in the direction of the incident/reflected light, the frequency of the backscattered or reflected light is altered by the Doppler effect. The Doppler frequency shift ΔF depends in particular on the amount and sign of the velocity component v_{z} of the moving object. In typical situations, ΔF ranges from about 10 kHz (for v_{z} in the order of cm/s and a light wavelength of 800 nm) to about 1000 kHz (for v_{z} in the order of m/s and a light wavelength of 800 nm).

The Doppler shifted radiation of frequency (f₀+F+ΔF) coming from the investigation region 3 is collected by the optics 14 and, after passing the frequency shifter 13 and the semi-reflective mirror 12, re-injected into the laser 11 (it should be noted that the preceding description is somewhat simplified; to keep a desirable automatic self-alignment of irradiation and detection, the light would have to pass the frequency shifter 13 twice with a shift by F/2 during each passage). As known from LOFT, the re-injected light causes oscillations of the laser intensity inside the laser 11. The amplitude of these oscillations critically depends on the frequency of the re-injected light.

FIG. 2 shows schematically the course G of the corresponding gain g of the oscillations in dependence on the difference (f−f₀) between the frequency f of re-injected light and the primary optical frequency f₀ (the exact gain function may be derived from equation (5) of Opt. Lett. 1999, pages 744-746). The gain g attains a maximal value gₘₐₓ if the re-injected light has a resonance frequency (f₀+Fᵣₑₗₐₓ), wherein Fᵣₑₗₐₓ is a relaxation frequency of the laser 11. In a standard LOFT sensor, the frequency shifter 13 is designed such that the primary optical frequency of the laser is shifted to said resonance frequency, i.e. F=Fᵣₑₗₐₓ.

Referring back to FIG. 1, a part of the light emitted by the laser 11 is reflected by the mirror 12 towards a detector 15, which is also (electronically) coupled to the frequency shifter 13. As is described in more detail in the cited literature, the detector 15 determines the disturbances introduced by the light re-injected into the laser 11, or, more particularly, determines the gain g of FIG. 2 that corresponds to the intensity oscillations.

An evaluator 16 (for example a microprocessor or a computer workstation with appropriate software) is coupled to the detector 15 and adapted to determine the velocity component v_{z} of the moving object 2 from the observed disturbances in the laser light. As can be seen from FIG. 2, the Doppler shifted re-injected radiation of frequency (f₀+F+ΔF) corresponds to a particular gain g(F,ΔF) of the intensity oscillations. Due to the known course of the gain function G in FIG. 2 and the known values of the primary frequency f₀ and the first frequency shift F introduced by the frequency shifter 13, the Doppler shift ΔF can be calculated from the measured value g(F,ΔF) by the evaluator 16. Based on this Doppler shift ΔF and on the known relations of the Doppler effect, the velocity component v_{z} of the moving object 2 can then be determined by the evaluator 16, too.

As can be seen from FIG. 2, the first frequency shift F introduced by the frequency shifter 13 is preferably set in such a way that the gap between F and the relaxation frequency Fᵣₑₗₐₓ is large enough to contain all expected or probable Doppler frequency shifts ΔF caused by moving objects. With other words, the maximal occurring Doppler frequency shift ΔFₘₐₓ is smaller than the difference (Fᵣₑₗₐₓ−F). Thus all measured gains g(F,ΔF) lie on the same (monotonous) branch of the gain function G. Remaining on the same branch of the gain function G guarantees a unique, invertible relation between gain g and Doppler frequency ΔF and thus allows the determination of the required velocity component v_{z} from the measured gain g(F,ΔF).

As is indicated by an arrow through the box of the frequency shifter 13 in FIG. 1, the first frequency shift F is preferably adjustable. Thus a range of frequencies may be scanned by varying the first shift frequency F. The maximal gain gₘₐₓ will then be observed if the sum of the currently set frequency shift F and the Doppler shift ΔF introduced by the moving object 2 corresponds to the relaxation frequency, i.e. if (F+ΔF)=Fᵣₑₗₐₓ. The advantage of this frequency-scanning approach is that the gain curve G of FIG. 2 must not be quantitatively known. Instead, it suffices to detect the occurrence of the maximal gain gₘₐₓ and to infer the Doppler shift ΔF from the currently set first frequency shift F by the formula ΔF=Fᵣₑₗₐₓ−F.

The optical path between the frequency shifter 13 and the optics 14 of the sensor 10 shown in FIG. 1 may for example be realized by fiber optics. Thus it is possible to build a short-range optical look ahead (SROLA) device for turbid media (like blood or tissue), which can be integrated into catheters, endoscopes or other interventional devices. This SROLA device is capable of imaging inside turbid media at distances of up to several mm with high spatial resolution. Thus planes positioned ahead of the current position of the device can be imaged, which is especially useful for navigation in high risk vascular structures (e.g. heart, brain). The device could be combined with e.g. fiber-optic confocal microscopes, OCT (Optical Coherence Tomography) catheters or other instruments for guidance.

Typically the SROLA device can be used for vascular interventions. The SROLA device will support navigation in complex vessel regions (bifurcations, lesions, chronic total occlusions). Critical parts of an intervention can be monitored in real-time (e.g.: embolization of aneurysms or stent positioning/deployment in coronary interventions).

Another important application of the invention is an enhanced surgical endoscope (ESE), which is a surgical endoscope equipped with a modified LOFT detector unit. This detector unit would preferably consist of an array of modified LOFT-detector/emitter elements added at the output of the endoscope. Such an ESE is able to provide visual information in cases where strong bleeding occurs and conventional endoscopes fail (e.g. neurosurgery).

The proposed catheter setup allows sectional scanning (like in OCT). Thus, the proposed method provides a means for optical sectional scanning without e.g. saline flushing. Due to the strong amplification of coherent signal photons, a significant penetration depth into biological tissue of several mm is possible. The technique is also applicable to other tissue surface layers (e.g. combination with photo-dynamic therapy (PDT), colon, skin). Finally, the modified LOFT detector of a catheter can serve as a local flow measurement tool. Data achieved before and after PTCA (percutaneous transluminal coronary angioplasty) or valve repair can for instance be compared against each other providing a means to verify the success of the procedure immediately after treatment without contrast agent and X-ray dose. Moreover, the sensor can be used to continuously monitor the velocity of a catheter relative to the vessel system or an organ, thus allowing a navigation on a (e.g. static) roadmap.

The spatial resolution of the described devices allows for the detection of structures exhibiting small scales, which is e.g. valuable for plaque assessment. Furthermore, a SROLA device would be hardly affected by artifacts (compare with IVUS: ringdown artifact near transducer, scattering from metal components like stents), resulting in a much better performance in e.g. stent imaging (PTCA outcome control). In addition, optical devices can be built very compact and at low cost. This makes the technology especially interesting in the domain of disposable devices.

Concerning the acquisition rate of the LOFT technique, 1 kHz have been shown to be the limit with a 1 mW laser at 1 μm wavelength and an effective reflectivity of only 2×10⁻¹³. The acquisition rate can be increased with increasing effective reflectivity or higher laser power.

FIGS. 3 and 4 show an exemplary embodiment of a scanning mechanism that may constitute a part of the optics 14 in FIG. 1 and that may be integrated into a typical catheter 100 (or catheter-like device). The mechanism comprises a mirroring element in the form of a planar, double-sided mirror 107 attached to two disk elements 106. The centre of the mirror 107 is always located on the optical axis z (or body axis) of the catheter 100 such that a radiation beam S traveling in optical fibers (not shown) along the catheter 100 will impinge on the mirror 107 from the direction of said axis. The beam S will then be reflected by the mirror 107 in a new direction according to the current orientation of the mirror.

The catheter 100 consists of three concentric tubes 101, 102, 103 which can be shifted relative to each other along the axial direction z. Moreover, the two inner tubes 102 and 103 can be rotated about said axis z with respect to the outer tube 101. The mirror 107 and the attached disk elements 106 are located inside the innermost tube 103. The contact zone between the disk elements 106 and the innermost tube 103 may be constructed as a tooth engagement mechanism with a tooth profile 104 on the inner catheter wall. Opposite to the contact zone between disk elements 106 and the innermost tube 103, said tube 103 comprises a window through which the disk elements 106 contact the middle tube 102. Again, the contact zone is constructed as a tooth engagement mechanism with a tooth profile 105.

If the innermost tube 103 is shifted axially in direction z relative to the middle tube 102, the mirror 107 is tilted about an axis x (FIG. 4) perpendicular to the body axis z. Moreover, if the two inner tubes 103 and 102 are commonly rotated about their axis z, the radiation beam S rotates about said axis z accordingly. By a superposition of a common rotation and a relative shift of the tubes 102 and 103, the light beam S can for example be scanned along a spiral path like that shown in FIG. 3.

Furthermore, the two inner tubes 102, 103 may be axially shifted with respect to the outer tube 101, thus moving the focal spot of the radiation beam S in z direction. The scanning mechanism therefore allows for a fast two- or three-dimensional scanning of a region ahead of the catheter tip.

Finally it is pointed out that in the present application the term “comprising” does not exclude other elements or steps, that “a” or “an” does not exclude a plurality, and that a single processor or other unit may fulfill the functions of several means. The invention resides in each and every novel characteristic feature and each and every combination of characteristic features. Moreover, reference signs in the claims shall not be construed as limiting their scope.

1. A Laser Optical Feedback Tomography (LOFT) sensor (10), comprising:
a laser source (11) for emitting a radiation beam at a primary optical frequency (f₀);
a frequency shifter (13) for shifting the primary optical frequency (f₀) of the radiation beam by a first frequency shift F,
optics (14) for irradiating an investigation region (3) of a medium (1) to be studied with radiation of the shifted frequency and for re-injecting into the laser (11) light sent back from the investigation region (3);
a detector (15) for detecting the disturbance brought to the laser (11) emission by the re-injected light;
an evaluator (16) coupled to the detector (15) and adapted to estimate the relative velocity (v_{z}) of moving objects (2) in the investigation region (3) based on the detected disturbances and the first frequency shift F.

2. The sensor (10) according to claim 1, characterized in that the evaluator (16) is adapted to estimate the relative velocity (v_{z}) of a moving object (2) based on a second frequency shift (ΔF) caused by said object in the light sent back from the investigation region (3).

3. The sensor (10) according to claim 1, characterized in that the frequency shifter (13) is adapted to selectively generate different shifting frequencies.

4. The sensor (10) according to claim 1, characterized in that the optics (14) is adapted to move the investigation region (3) within the medium (1) to be studied.

5. A minimal invasive interventional instrument, particularly a catheter (100) or an endoscope, which is provided with a modified LOFT sensor (10) according to claim 1.

6. A method for the determination of the velocity (v_{z}) of an object (2) relative to an instrument (100), comprising the steps of
providing the instrument (100) with a LOFT sensor comprising a laser (11), particularly a LOFT sensor (10) according to claim 1;
irradiating the object (2) with radiation that is shifted from the primary optical frequency (f₀) of the laser (11) by a first frequency shift F,
re-injecting into the laser (11) light sent back from the investigation region (3);
detecting the disturbance brought to the laser (11) emission by the re-injected light;
estimating the relative velocity (v_{z}) of the object (2) in the investigation region (3) based on the detected disturbances and the first frequency shift F.

7. The method according to claim 6, characterized in that the velocity (v_{z}) of the object (2) is determined based on a second frequency shift ΔF caused by said object in the light sent back from the investigation region (3).

8. The method according to claim 7, characterized in that the first frequency shift F is chosen such that expected second frequency shifts ΔF caused by objects (2) are smaller than a frequency gap between the first frequency shift F and a corresponding relaxation frequency Fᵣₑₗₐₓ Of the laser (11).

9. The method according to claim 6, characterized in that the first frequency shift F is scanned through a predetermined range.

10. The method according to claim 6, characterized in that the instrument (100) is navigated relative to the object (2).

11. A scanning mechanism for selectively directing a radiation beam (S) from an interventional instrument (100) into a surrounding medium (1), comprising a remotely movable mirroring element (106, 107) arranged at the light outlet of the instrument (100).

12. The mechanism according to claim 11, characterized in that the mirroring element (106, 107) can be shifted along the propagation axis (z) of the incident light and/or rotated about this axis (z) and/or rotated about an axis (x) perpendicular thereto.

13. The mechanism according to claim 11, characterized in that the mirroring element (106, 107) is mounted between two carriers (102, 103) that can be axially shifted relative to each other.

14. The mechanism according to claim 13, characterized in that the carriers (102, 103) can be commonly rotated about their axis (z).

15. The mechanism according to claim 13, characterized in that the carriers are constituted by concentric tubes (102, 103) which are preferably embedded in an outer tube (101).
